SEQUENCE LISTING (6 sequences)

SEQ ID NO: 1 - DNA, 28 bp, Fusarium oxysporum
agcactagtaatctacccgaggccagtc

SEQ ID NO: 2 - DNA, 28 bp, Fusarium oxysporum
aggcaattgggcgattttgacgttgaga

SEQ ID NO: 3 - DNA, 27 bp, Magnaporthe grisea
acggaattctcgacatggaccgtcttg

SEQ ID NO: 4 - DNA, 27 bp, Magnaporthe grisea
agctctagagtaccaagccgcaccact

SEQ ID NO: 5 - DNA, 29 bp, Escherichia coli
tcgctctagaaataatgattttatttgac

SEQ ID NO: 6 - DNA, 30 bp, Escherichia coli
tcgcaagcttgctggatggcaaataatgat

(19) United States
(12) Patent Application Publication
(10) Pub. No.: US 2005/0181509 A1
(43) Pub. Date: Aug. 18, 2005

(54) Dual selection based, targeted gene disruption method for fungi and fungus-like organisms

(75) Inventors: Seogchan Kang, Port Matilda, PA (US); Chang Hyun Khang, State College, PA (US)

Correspondence Address: MCKEE, VOORHEES & SEASE, P.L.C.;ATTN: PENNSYLVANIA STATE UNIVERSITY, 801 GRAND AVENUE, SUITE 3200, DES MOINES, IA, 50309-2721, US

(73) Assignee: The Penn State Research Foundation, University Park, PA (US)

(21) Appl. No.: 10/777,405

(22) Filed: Feb. 12, 2004

(51) Int. Cl. C12N 15/74; C12N 1/16

(52) U.S. Cl. 435/483; 435/254.2

(57) ABSTRACT

The invention disclosed herein is useful as an efficient targeted gene manipulation tool that can be applied, with minimal modifications, to targeted genes in a broad spectrum of fungi and fungus-like organisms. The invention is based on Agrobacterium tumefaciens-mediated transformation followed by a subsequent positive-negative selection scheme to isolate target mutants.

### GRANT REFERENCE

Work for this invention was funded in part by a grant from the USDA under the Hatch Act for Project No. PEN03652. The Government may have certain rights in this invention.

### BACKGROUND OF THE INVENTION

Fungi have a far-reaching influence on our lives. As recyclers of organic matter or as root symbionts of most terrestrial plants, many fungi are essential components of a healthy ecosystem. Some fungi have been extensively utilized for the production of useful compounds, including pharmaceuticals, organic acids, industrial enzymes and recombinant proteins (Demain, 2000; Askenazi et al., 2003). Considering the diverse metabolic capacities in a limited number of fungi that have been commercially utilized, the fungal kingdom represents a vastly under-explored resource (Hawksworth, 1991) for many more valuable compounds. In contrast to these benefits, fungi that have evolved the ability to exploit other organisms via pathogenic associations often cause devastating diseases in plants and/or animals (Hudler, 1998). Fungal diseases are by far the most serious threat to global crop production, and possess the ability to inflict enormous losses that can result in serious socioeconomic hardship. Fungi also present a direct threat to human health, as one of the most common causes of death in immune-compromised patients. Most fungi and fungus-like organisms (such as oomycetes) of practical significance have not been well characterized due to a number of factors, including the lack of efficient tools for manipulating their genes. Development of such tools is essential for efficient use of the growing body of genomic sequence data from fungi and fungus-like organisms. A better understanding of fungal biology will not only facilitate judicious use of beneficial fungi, but also advance efforts to develop effective measures for controlling pathogenic fungi. Such an understanding has greatly increased in recent years, due in large part to the application of molecular tools. Among these tools, transformation-mediated mutagenesis and complementation analyses have undoubtedly been the most widely applied methods for studying gene function in fungi. In most filamentous fungi, transformation results from the integration of the transforming DNA into the fungal genome by either illegitimate or homologous recombination. Homologous integration permits targeted gene disruption.

Targeted gene disruption is a particularly productive approach to understanding the function of a particular gene in an organism and involves the disruption of the gene's function which is colloquially referred to as a “targeted mutagenesis”. One common form of targeted mutagenesis is to generate “gene knockouts”. Typically, a gene knockout involves disrupting a gene in the genome of an organism. Once established in the genome of the organism, it is possible to determine the effect of the mutation on the organism.

The most common approach to producing knockout organisms involves the disruption of a target gene by inserting into the target gene, via homologous recombination, a DNA construct encoding a selectable marker gene flanked by DNA sequences homologous to part of the targeted gene. When properly designed, the DNA construct effectively integrates into and disrupts the targeted gene, thereby preventing expression of an active gene product encoded by that gene. Homologous recombination involves recombination between two genetic elements (either extrachromosomally, intrachromosomally, or between an extrachromosomal element and a chromosomal locus) via homologous DNA sequences, which results in the physical exchange of DNA between the genetic elements. Homologous recombination is not limited to mammalian cells but also occurs in bacterial cells, fungal cells, in the slime mold Dictyostelium discoideum and in other organisms. For a review of homologous recombination in fungal cells, see Orr-Weaver et al., Microbiol. Reviews, 49:33-58 (1985) incorporated herein by reference.

Although a number of techniques have been employed to manipulate genes in fungi and fungus-like organisms, those based on transformation are by far the most commonly used. In most fungi and fungus-like organisms, transformation typically results in either the heterologous integration or the homologous integration of introduced DNA into the genome. Gene replacement via homologous recombination, in which the chromosomal, wild-type copy of a gene is replaced with a mutant allele introduced by transformation, has been widely used to function with this technique in fungi and fungus-like organisms, but has been plagued by a low frequency of homologous integration. Unfortunately, unlike yeast Saccharomyces cerevisiae, in many fungi and fungus-like organisms, transformation mainly occurs via heterologous integration of introduced DNA. This necessitates a large number of transformants to be generated, purified (through single spore isolation and/or serial transfer) and screened (by polymerase chain reaction or Southern analysis) in order to identify the desired mutant. Agrobacterium tumefaciens mediated transformation (ATMT) has been used to manipulate genes in fungi and fungus-like organisms for several years. Although ATMT offers a number of advantages over conventional transformation techniques in gene manipulations, further improvement of the technique is needed to expedite large-scale functional genomic analyses of fungi and fungus-like organisms. Pratt et al., Fungal Genetics and Biology 37:56-71 (2002) discloses the use of the mating type heterokaryon incompatibility system as a counter-selection to increase the probability of identifying gene replacement in Neurospora crassa, which employs a double selection system. While this technique allows a significant enrichment of gene knockout mutants, its utility is limited because the negative selection marker used, the mat α-1 gene, confers toxicity only to N. crassa. It can be seen from the foregoing that a need exists to circumvent the time-consuming process of regenerating and screening a large number of transformants to identify desired gene disruptants in fungi that exhibit low frequencies of homologous integration. Therefore, it is a primary object, feature, or advantage of the present invention to improve upon the state of the art.

It is a further object, feature, or advantage of the invention to provide a highly efficient tool for the identification and selection of transformants that is widely-applicable in diverse fungi and fungus-like organisms.

A further object, feature, or advantage of the invention is to provide vehicles for transforming fungal cells, such as plasmid vectors incorporating a construct comprising a negative selection marker linked to a DNA fragment flanked by sequences homologous to part of the target gene that is disrupted by the insertion of a positive selection marker.

A further object, feature, or advantage of the invention is to provide fungal cells comprising such vectors.

It is yet another object, feature, or advantage of the invention to provide expression constructs for transforming fungal host cells which provide for creation of transformants.

Another object, feature, or advantage of the invention is to provide for a screening method to select for transformed mutants.

These and other objects, features, or advantages will become apparent from the following description of the invention.

### BRIEF SUMMARY OF THE INVENTION

This invention relates to providing a novel targeted gene manipulation tool, which is based on the combination of a transformation method which allows for homologous recombination between targeting constructs and any DNA segment of the fungi, fungus-like organism or other eukaryotic genome, and a subsequent positive-negative selection scheme. Various methods have been developed to facilitate the transformation of fungi and fungus-like organisms and offer one or more of the following advantages, including high efficiency of transformation, increased frequency of homologous recombination, ability to transform spores, hyphae, and protoplasts, and low copy number of inserted DNA per genome. Applicants have improved upon the state of the art by developing a subsequent positive-negative selection scheme that permits the rapid isolation of desired mutants even when the frequency of homologous recombination is low, thus maximizing the benefits of Agrobacterium tumefaciens-mediated transformation (ATMT). The present invention can be applied to phylogenetically diverse fungi and fungus-like organisms with minimal modifications, because most of the positive and negative selection markers chosen can function in diverse fungi and fungus-like organisms.

According to the present invention, homologous recombination allows the preparation of constructs to target essentially any DNA segment of the fungi, fungus-like organism genome, or other eukaryotic genome. The constructs of the present invention comprise targeting DNA sequences or DNA fragments which are homologous to one or more portions of a gene or genetic locus to be targeted. Targeting constructs may further comprise disrupter elements (such as marker genes) within the targeting DNA sequences which when introduced into the targeted gene or locus (hereinafter the “target” or “targeted DNA”) by way of homologous recombination, disrupts the expression of the targeted DNA. In addition, a negative selection marker is added. The negative selection marker is outside the region of the sequence of similarity between the vector and the targeted gene or locus. This approach is exemplified below with reference to particular polynucleotide sequences and particular fungal strains, however, the methods of the present invention are readily adaptable to other polynucleotide sequence and other species of fungi and other eukaryotic genomes. Alternatively, instead of a disruptor element, a transcriptional regulatory sequence or another gene or portion thereof may be flanked by homologous targeting sequences, thereby allowing their introduction into a specific gene or genetic locus. Such alternative constructs may also comprise a marker gene in an orientation that allows its expression but does not disrupt the function of the target gene.

Targeting constructs may also comprise replication competent or deficient vectors such as plasmids, phagemids, cosmids, artificial yeast chromosomes, and viruses such as bacteriophage or mammalian viruses. The use of replication incompetent vectors may require the coincident use of helper viruses or other helper elements which complement the replication defect in the vector.

Cells preferred as hosts for the practice of the invention include those cells competent to mediate homologous recombination, that is cells that permit recombination between homologous DNA sequences on the same genetic element or between separate genetic elements. Preferred cells include fungi including yeast, insect cells, amphibian cells, slime molds, and bacterial cells. Most preferred are filamentous fungi cells and, in particular, Magnaporthe grisea and Fusarium oxysporum.

Essential nucleic acid molecules of the present invention are contained within the targeting DNA or DNA fragment and may be any polynucleotide sequence or coding region that expresses a detectable phenotype. The term “essential gene” or “essential nucleic acid molecule” means the polynucleotide sequence that is necessary to display a specific characteristic in the cell. For an example, “essential nucleic acid molecule” for growth or environmental conditions means that cells are not viable if the nucleic acid molecule is disrupted or if cells are grown under a specified set of conditions that require its expression. The “essential nucleic acid molecule” used in the context of a phenotype means that cells do not display the specific phenotype if the essential nucleic acid molecule is disrupted or prevented from expression. Essential nucleic acid molecules are contained within “target DNA”. “Target DNA” may be any DNA that contains the essential nucleic acid molecule. It may be, for example, restricted chromosomal or genomic DNA or may be a short gene fragment. Essential nucleic acid molecules of the present invention are contained within the chromosomal DNA fragments and may be any polynucleotide sequence or coding region that expresses a detectable phenotype. Typically, the essential nucleic acid molecule will be present in the host organism. However, the present method is applicable to situations where the essential nucleic acid molecule is only a homolog of one in the host genome. In some instances the essential nucleic acid molecule may be essential for cell growth under any conditions. In this case, disruption of this essential nucleic acid molecule will lead to cell death. More typically, the essential nucleic acid molecule will encode an enzyme necessary for growth under specific conditions, i.e., amino acid synthesis. Examples of specific phenotypes that may be screened for in the present method include but are not limited to, metabolic capacity (e.g., carbon source requirement, amino acid requirement, nitrogen source requirement, and purine requirement); resistance to inorganic chemicals (e.g., acid, arsenate, azide, heavy metals, and peroxide); resistance to organic and biological chemicals (e.g., herbicides, fungicides, bactericidal agents, bacteriostatic agents, antibiotics, acridine, actinomycin, amino purine, amino phenylalanine, colicin, ethanol, fluoroacetate, mitomycin C, and nalidixic acid); resistance to biological agents (e.g., phages); resistance to physical extremes (e.g., temperature, pH, osmotolerance and radiation); enzymatic function (e.g., assays for protease, phosphatase, coagulase, urease, catalase, etc.); fatty acid composition; degradation; and hydrolysis. The phenotypes amenable to detection by the present invention are numerous and are contemplated by this invention.

One embodiment of the invention provides a method of identifying and selecting transformants comprising transforming a host cell with Agrobacterium under suitable conditions whereby recombination occurs, the Agrobacterium comprising a vector containing a targeting construct wherein said construct comprises a first polynucleotide sequence encoding a negative selection marker linked to a fragment of DNA flanked by DNA sequences homologous to the polynucleotide to be targeted, wherein said DNA fragment is disrupted by a positive selection marker, which confers resistance to an antibiotic; and selecting transformants by subjecting a transformed host cell to both a positive and a negative selection agent.

Another embodiment of the invention provides a method of identifying a knockout mutant comprising (a) providing a polynucleotide construct comprising a first polynucleotide sequence that encodes a negative selection marker linked to a fragment of genomic DNA flanked by DNA sequences homologous to the gene to be targeted, wherein said DNA fragment is disrupted by a positive selection marker; (b) introducing into Agrobacterium the construct provided in (a), thereby producing a resultant Agrobacterium cells containing a DNA fragment with a disrupted sequence; (c) incubating Agrobacterium produced in (b) with fungal cells under conditions so that T-DNA containing said construct is integrated into a fungal cell genome, wherein transformants resulting from knockout lack a negative selection marker and ectopic, heterologous, or illegitimate transformants express both a negative and a positive selection marker; and (d) selecting knockout mutants by subjecting transformed fungal cells to a positive and a negative selection agent.

Yet another embodiment of the invention provides a method of transforming fungal cells to identify mutants comprising inserting a polynucleotide construct to be introduced into fungal cells into an Agrobacterium-based vector between T-DNA borders in that vector; introducing said vector containing the DNA construct into Agrobacterium tumefaciens cells, wherein the cells contain a virulence region in its DNA; inducing virulence genes to T-DNA containing the construct from the Agrobacterium tumefaciens and incubating the Agrobacterium tumefaciens with a fungal cells to be transformed; and selecting transformed fungal cells from untransformed fungal cells by subjecting transformants to a positive and a negative selection agent.

One or more of these and/or other objects, features, or advantages of the present invention will become apparent from the specification and claims that follow.

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art.

Various units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5th edition, 1993). The terms defined below are more fully defined by reference to the specification as a whole.

In describing the present invention, the following terms will be employed, and are intended to be defined as indicated below.

Definitions

As used herein the term “Agrobacterium” shall be intended to include any bacterial species and its conservatively modified variants that are capable of infecting a desired fungal cell. The Agrobacterium tumefaciens Ti plasmid is described herein, but the invention is not so limited. The choice of particular bacterial vector involves no more than routine optimization of parameters by those of skill in the art. Other bacteria may be used and are available to those of skill in the art through sources such as GenBank.

A “cloning vector” is a DNA molecule such as a plasmid, cosmid, or bacterial phage that has the capability of replicating autonomously in a host cell. Cloning vectors typically contain one or a small number of restriction endonuclease recognition sites at which foreign DNA sequences can be inserted in a determinable fashion without loss of essential biological function of the vector, as well as a selection marker that is suitable for use in the identification and selection of cells transformed with the cloning vector. Selectable markers typically include those that provide resistance to antibiotics such as hygromycin, neomycin, or kanamycin.

A “coding sequence” or “coding region” refers to a nucleic acid molecule having sequence information necessary to produce a gene product, when the sequence is expressed.

The term “conservatively modified variants” applies to both amino acid and nucleic acid sequences. With respect to particular nucleic acid sequences, conservatively modified variants refer to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid that encodes a polypeptide of the present invention is implicit in each described polypeptide sequence and is within the scope of the present invention.

As to amino acid sequences, one of skill will recognize that individual substitutions, deletions, or additions to a nucleic acid, peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).
   See also, Creighton (1984) Proteins, W.H. Freeman and Company.

By “encoding” or “encoded”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code.

The term “expression” refers to biosynthesis of a gene product. Structural gene expression involves transcription of the structural gene into mRNA and then translation of the mRNA into one or more polypeptides.

An “expression vector” is a DNA molecule comprising a gene that is expressed in a host cell. Typically, gene expression is placed under the control of certain regulatory elements including promoters, tissue specific regulatory elements, and enhancers. Such a gene is said to be “operably linked to” the regulatory elements.

The phrase “hybridizes under stringent conditions” refers to the formation of a double-stranded duplex by two single-stranded nucleic acids. The region of double-strandedness can include the full-length of one or both of the single-stranded nucleic acids, or all of one single stranded nucleic acid and a subsequence of the other single stranded nucleic acid, or the region of double-strandedness can include a subsequence of each nucleic acid. An extensive guide to the hybridization of nucleic acids is found in Tijssen, Laboratory Techniques in Biochemistry and Molecular Biology—Hybridization with Nucleic Acid Probes Parts I and II, Elsevier, N.Y., (1993). Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Highly stringent conditions are selected to be equal to the Tm point for a particular probe.

An example of stringent hybridization conditions for hybridization of complementary nucleic acids that have more than 100 complementary residues on a filter in a Southern or northern blot is 50% formalin with 1 mg of heparin at 42° C., with the hybridization being carried out overnight. An example of stringent wash conditions for a Southern blot of such nucleic acids is a 0.2×SSC wash at 65° C. for 15 minutes (see, Sambrook, et al., Molecular Cloning—A Laboratory Manual (2nd ed.) Vol. 1-3, Cold Spring Harbor Laboratory, Cold Spring Harbor Press, New York, 1989 (Sambrook) for a description of SSC buffer). Often the high stringency wash is preceded by a low stringency wash to remove background probe signal. An example of low stringency wash is 2×SSC at 40° C. for 15 minutes. In general, a signal to noise ratio of 2×(or higher) than that observed for an unrelated probe in the particular hybridization assay indicates detection of a specific hybridization. For highly specific hybridization strategies such as allele-specific hybridization, an allele-specific probe is usually hybridized to a marker nucleic acid (e.g., a genomic nucleic acid, or the like) comprising a polymorphic nucleotide under highly stringent conditions. “Nucleic acid sequence homologs” refers to deoxyribonucleotides or ribonucleotides and polymers thereof in either single- or double-stranded form containing known analogs of natural nucleotides, which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer, et al., Nucleic Acid Res. 19:5081 (1991); Ohtsuka, et al., J. Biol. Chem. 260:2605-2608 (1985); and Rossolini, et al., Mol. Cell. Probes 8:91-98 (1994)).

By “host cell” is meant a cell that contains a vector and supports the replication and/or expression of the vector. Host cells may be prokaryotic cells such as E. coli, or eukaryotic cells such as fungi, insect, or amphibian cells. Preferably, the host cells are filamentous fungi. “Fungi” as used herein includes the phyla Ascomycota and Basidiomycota. By “fungus-like organisms”, it is meant the phyla Oomycota.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The term “transformation” refers to the introduction of a transgene into a fungal cell, either in culture or into the tissues of fungi by a variety of techniques used by molecular biologists. A number of techniques are known in the art for transformation of fungus or fungi-like organism in general, including Agrobacterium-mediated transformation, electroporation, microinjection, microprojectile or particle gun technology (biolistics), liposomes, polyethylene glycol (PEG) mediated transformation, wounding, vacuum infiltration, passive infiltration or pressurized infiltration, and reagents that increase free DNA uptake.

The term “polynucleotide construct” or “DNA construct” is defined herein as a nucleic acid molecule, either single- or double-stranded, which has been modified to contain segments of nucleic acid combined and juxtaposed in a manner that would not otherwise exist in nature. These terms are synonymous with the term expression cassette or sometimes used to refer to an expression construction, when the nucleic acid construct contains a coding sequence and all the control sequences required for expression of the coding sequence.

The term “operably linked” means that the regulatory sequences necessary for expression of the coding sequence are placed in a nucleic acid molecule in the appropriate positions relative to the coding sequence so as to enable expression of the coding sequence. This same definition is sometimes applied to the arrangement of other transcription control elements (e.g., enhancers) in an expression vector.

Transcriptional and translational control sequences are DNA regulatory sequences, such as promoters, enhancers, polyadenylation signals, terminators, and the like, that provide for the expression of a coding sequence in a host cell.

As used herein, “polynucleotide” includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons as “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein that protein is specifically reactive to antibodies elicited to the same protein but consisting entirely of naturally occurring amino acids. The terms “polypeptide”, “peptide” and “protein” are also inclusive of modifications including, but not limited to, phosphorylation, glycosylation, lipid attachment, sulfation, gamma-carboxylation of glutamic acid residues, hydroxylation and ADP-ribosylation. It will be appreciated, as is well known and as noted above, that polypeptides are not entirely linear. For instance, polypeptides may be branched as a result of ubiquitination, and they may be circular, with or without branching, generally as a result of post translation events, including natural processing event and events brought about by human manipulation, which do not occur naturally. Circular, branched, and branched circular polypeptides may be synthesized by a non-translation natural process and by entirely synthetic methods as well. Further, this invention contemplates the use of both the methionine-containing and the methionine-less amino terminal variants of the protein of the invention. With respect to a protein, the term “N-terminal region” shall include approximately 50 amino acids adjacent to the amino terminal end of a protein.

The terms “promoter”, “promoter region”, or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase. The term promoter includes the essential regulatory features of said sequence and may optionally include a long terminal repeat region prior to the translation start site.

With respect to oligonucleotides or other single-stranded nucleic acid molecules, the term “specifically hybridizing” refers to the association between two single-stranded nucleic acid molecules of sufficiently complementary sequence to permit such hybridization under pre-determined conditions generally used in the art and discussed herein, i.e., conditions of stringency (sometimes termed “substantially complementary”). In particular, the term refers to hybridization of an oligonucleotide with a substantially complementary sequence contained within a single-stranded DNA or RNA molecule, to the substantial exclusion of hybridization of the oligonucleotide with single-stranded nucleic acids of non-complementary sequence.

A “vector” is a replicon, such as plasmid, phage, cosmid, or virus to which another nucleic acid segment may be operably inserted so as to bring about the replication or expression of the segment.

The term “gene targeting” refers to a type of homologous recombination that occurs when a fragment of genomic DNA is introduced into a host cell and that fragment locates and recombines with endogenous homologous sequences.

The term “homologous recombination” refers to the exchange of DNA fragments between two DNA molecules or chromatids at the site of homologous nucleotide sequences.

The term “homologous” as used herein denotes a characteristic of a DNA sequence having at least about 70 percent sequence identity as compared to a reference sequence, typically at least about 85 percent sequence identity, preferably at least about 95 percent sequence identity, and more preferably about 98 percent sequence identity, and most preferably about 100 percent sequence identity as compared to a reference sequence. Homology can be determined using a “BLASTN” algorithm. It is understood that homologous sequences can accommodate insertions, deletions and substitutions in the nucleotide sequence. Thus, linear sequences of nucleotides can be essentially identical even if some of the nucleotide residues do not precisely correspond or align. The reference sequence may be a subset of a larger sequence, such as a portion of a gene or flanking sequence, or a repetitive portion of a chromosome.

The term “target gene” (alternatively referred to as “target gene sequence” or “target DNA sequence” or “target sequence” or “target sequence of interest”) refers to any nucleic acid molecule or polynucleotide of any gene to be modified by homologous recombination. The target sequence may include an intact polynucleotide sequence, an exon or intron, a regulatory sequence or any region between genes.

“Disruption” of a polynucleotide sequence occurs when a positive selection marker is inserted into a DNA fragment. These sequence disruptions or modifications may include insertions, missense, frameshift, deletion, or substitutions, or replacements of DNA sequence, or any combination thereof.

As used herein, the term “positive selection” refers to the case in which a host cell grown in the presence of a positive selective agent such as hygromycin B and geneticin or G-418 can survive only when the cells containing the positive selectable marker gene such as the hygromycin B phosphotransfererase (hph) gene or neomycin phosphotransferase (npt) gene, respectively, replicates within the cell, and the hph or npt gene is expressed. Other positive markers include, but are not limited to, mutated beta-tubulin (ben) gene, which confers resistance to benomyl; Bar, which confers resistance to basta; Ble, which confers resistance to phleomycin; Sat-1, which confers resistance to nourseothricin, and cbx, conferring resistance to carboxin. Genes essential for the synthesis of an essential nutrient (such as amino acid arginine and nucleoside phrimidine) may also be used as positive selection markers and are contemplated by the present invention. To use such markers, the fungal strain to be transformed should have a mutation in these genes.

As used herein, the term “negative selection” refers to the situation in which a host cell grown in the presence of a negative selective agent such as acyclovir, ganciclovir, or 5-fluoro-2′-deoxyuridine (F2dU) dies if the cell containing a suicide gene, such as the herpes simplex virus (HSV) thymidine kinase (TK), replicates within the cell, and the TK gene is expressed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram of ATMT-PNS. Agrobacterium tumefaciens cells, carrying a binary vector that contains a mutant allele (disrupted by a positive selection marker, such as hph, marked as the filled box) and HSVtk (denoted by the diamond) on the T-DNA, are co-incubated with fungal cells in the presence of acetosyringone (AS), a chemical inducer of virulence genes of A. tumefaciens. During co-cultivation, DNA situated between the left border (LB) and right border (RB) of the T-DNA is transported into fungal nuclei. Homologous recombination between the native gene and the mutant allele on the T-DNA leads to the loss of HSVtk. If the T-DNA integrates into a random location in the fungal genome via illegitimate recombination, both hph and HSVtk will be expressed. Gene KO mutants can be selected by subjecting transformants to both the positive (hygromycin B) and negative (F2dU) selection agents.

FIG. 2 is a schematic diagram of the T-DNA of the binary vectors used in this study. The LB and RB of the T-DNA are denoted by vertical lines. The orientation of transcription from hph, neo and HSVtk is indicated by arrow (5′ to 3′). MCS1 corresponds to the multiple cloning site of pCAMBIA1300. The multiple cloning site of pGreenII0000 cloned in pDHt was designated as MCS-SK or MCS-KS depending on its orientation: KpnI (K) and SacI (Sc) sites are shown to indicate the orientation of the MCS relative to other markers. A modified version of HSVtk via site-directed mutagenesis is denoted as HSVtk(M). Gateway corresponds to the ccdB and chloramphenicol-resistance genes flanked by the λattP sites.

FIG. 3 shows the growth of Magnaporthe grisea, Fusarium oxysporum, Aspergillus fumigatus, and Botrytis cinerea in the presence of F2dU or Ganciclovir. Wild-type strains (wt) and transformants with ChGPD-HSVtk (tk) of M. grisea (A & E), F. oxysporum (B), A. fumigatus (C), and B. cinerea (D) were grown in the presence of F2dU (A-D) or Ganciclovir (E) at concentrations ranging from 5 nM to 50 μM (F2dU) or 1 μM to 2 mM (Ganciclovir).

FIG. 4 shows a Southern analysis of selected M. grisea transformants. The hatched box interrupted by hph denotes the mhp 1 mutant allele cloned in pGKO1. EcoRI-digested genomic DNA of wild type 4091-5-8 strain (lane 1) and its transformants, including one gene knockout (KO) mutant (lane 2), one ectopic transformant (lane 3), and three different types of FPs (lanes 4-6), was hybridized with each of the four probes shown underneath the T-DNA diagram: (A) 0.3 kb fragment covering the region between the LB and the ChGPD promoter, (B) 0.4 kb fragment covering the ChGPD promoter, (C) 2.9 kb fragment covering both hph and parts of the MHP1 locus, (D) 250 bp fragment covering the region between the RB and the mutant allele. The arrow in panel C marks the wild-type MHP1 gene, which was absent in the gene KO mutant (lane 2).

FIG. 5 shows stability of neo and HSVtk at the LB and RB sides. Each fungal strain was transformed using (A) pNHTK and (B) pTKHN. The total number of hygromycin B-resistant transformants analyzed (HR), and the number and percentage of HR sensitive to geneticin and F2dU (loss of neo), resistant to F2dU and geneticin (loss of HSVtk), and sensitive to geneticin and resistant to F2dU (loss of both markers) were indicated in the tables.

The following sequences comply with 37 C.F.R. 1.821-1.825 (“Requirements for Patent Applications Containing Nucleotide Sequences and/or Amino Acid Sequence Disclosures—the Sequence Rule”) and are consistent with World Intellectual Property Organization (WIPO) Standard ST.25 (1998) and the sequence listing requirements of the EPO and PCT (Rules 5.2 and 49.5 (a-bis), and Section 208 and Annex C of the Administrative Instructions). The symbols and format used for nucleotide and/or amino acid sequence comply with the rules set forth in 37 C.F.R. § 1.822.

SEQ ID NO: 1-6 are the DNA primer sequences used in the present invention.

All references cited herein are hereby incorporated in their entirety by reference.

### DETAILED DESCRIPTION OF THE PREFERRED EMBODIMENT

Homologous recombination relies on the tendency of nucleic acids to base pair with complementary sequences. In this instance, the base pairing serves to facilitate the interaction of two separate nucleic acid molecules so that strand breakage and repair can take place. In other words, the “homologous” aspect of the method relies on sequence homology to bring two complementary sequences into close proximity, while the “recombination” aspect provides for one complementary sequence to replace the other by virtue of the breaking of certain bonds and the formation of others.

Put into practice, homologous recombination in the context of the present invention is used as follows. First, a target gene is selected within the host cell. Sequences homologous to the target gene are included in a polynucleotide construct. Typically, the portion of the gene included in the targeting construct is interrupted by insertion of a marker sequence (usually a selectable marker) that disrupts the reading frame of the interrupted gene so as to preclude expression of an active gene product. This most often causes a knock out or inactivation of a gene. The homologous sequences on either side of the modifying mutation are said to “flank” the mutation. Flanking, in this context, simply means that target homologous sequences are located both upstream (5′) and downstream (3′) of the mutation. These sequences should correspond to some sequences upstream and downstream of the target gene. The construct is then introduced into the host cell, thus permitting recombination between the genomic sequences and the construct. Targeted mutagenesis of a gene will result in an alteration (e.g., partial or complete inactivation or constitutively) of normal production or structure of the polypeptide encoded by the targeted gene of a single cell, selected cells or all of the cells in culture by introducing an appropriate targeting construct into a site in the gene to be disrupted.

Another refinement of the homologous recombination approach involves the use of a “negative” selectable marker. This marker, unlike the selectable marker, causes death of cells which express the marker. Thus, it is used to identify undesirable recombination events. When seeking to select homologous recombinants using a selectable marker, it is difficult in the initial screening step to identify proper homologous recombinants from recombinants generated from random, non-sequence specific events. These recombinants also may contain the selectable marker gene, but will, in all likelihood, not have the desired “knock out” phenotype. By attaching a negative selectable marker to the construct, one can select against many random recombination events that will incorporate the negative selectable marker. Homologous recombination will likely not introduce the negative selectable marker, as it is outside of the flanking sequences.

Thus, for preparing knockouts, a gene within a host cell is chosen as the target gene into which a selection marker gene is to be transferred. Sequences homologous to the target gene are included in the disruption vector, and the selection gene is inserted into the vector such that target gene homologous sequences are interrupted by the selection marker. Applicants have found application of a subsequent positive-negative selection permits the rapid isolation and identification of desired mutants even when the frequency of homologous recombination is low.

According to the present invention, homologous recombination in fungi and fungus-like organisms allows the preparation of targeting constructs to target essentially any segment of the fungal or fungus-like organism or other eukaryotic genome.

Nucleotide sequences may be introduced into the host cell by any method known to one skilled in the art. Transformation techniques such as the use of microinjection, microprojectile-bombardment, electroporation and others known to the skilled man are among those methods for which this invention is appropriate. Additional methods include bacterial infection (e.g., with Agrobacterium as described below), binary bacterial artificial chromosome constructs, and desiccation/inhibition-mediated DNA uptake (reviewed in Potrykus, Ann. Rev. Plant Physiol. Plant Mol. Biol., 42:205, 1991). In a preferred embodiment of the present invention, the Agrobacterium-Ti plasmid system is utilized.

For review articles on the transformation of fungi reference is made to the articles:

“Transformation in Fungi” by John R. S. Fincham published in Microbiological Reviews (March 1989) 148-170, which gives an outline of the possible transformation methods for fungi, i.e. both yeasts and moulds.

“Genetic engineering of filamentous fungi” by Timberlake, W. E. and Marshall, M. A. Science 244 (1989) 1313-1317.

“Transformation” by David B. Finkelstein (Chapter 6 in the book “Biotechnology of Filamentous Fungi, Technology and Products” (1992) 113-156, edited by Finkelstein and Ball).

From this literature it is clear that several transformation techniques have been developed to transform an increasing number of filamentous fungi. Most transformation protocols make use of protoplasts. Protoplasts can be prepared from hyphal cultures or germinating conidia using Novozyme 234^{R}, a multi-enzyme preparation derived from Trichoderma reesei. Transformation of protoplasts with DNA is mediated by electroporation or by a combination of CaCl₂ and polyethylene glycol (PEG). Some alternative methods avoid the need for making protoplasts, which renders the procedure more rapid and simpler. Intact cells can be transformed using a combination of lithium acetate and PEG, particle bombardment (Lorito et al.; Curr. Genet. 24 (1993) 349-356 and Herzog et al.; Appl. Microbiol. Biotechnol. 45 (1996) 333-337) or also electroporation (Ozeki et al.; Biosci. Biotech. Biochem. 58 (1994) 2224-2227).

Plant transformation using Agrobacterium

A transformation technique developed for plants is based on the use of Agrobacterium tumefaciens, which is a gram-negative soil bacterium that causes crown gall tumors at wound sites of infected dicotyledonous plants. During tumor induction Agrobacterium attaches to plant cells and then transfers part of its tumor-inducing (Ti) plasmid, the transferred DNA or T-DNA, to the cell where it becomes integrated in the plant nuclear genome. The T-DNA is flanked by 24 basepair imperfect direct repeats. These direct repeats are also known as “border repeats” or “borders” or “T-DNA borders” or “border sequences” or combinations thereof. The T-DNA contains a set of genes. Expression of a subset of these genes, the onc genes, leads to the production of phytohormones which induce plant cell proliferation and the formation of a tumor. The process of transfer depends on the induction of a set of virulence genes encoded by the Ti plasmid. The transfer system is activated when VirA senses inducing compounds from wounded plants, such as acetosyringone (AS). Via the transcriptional activator VirG, the remaining vir loci are activated and a linear single-stranded DNA, the T-strand, is produced following nicking of the border repeats by a virD1/D2 encoded site-specific endonuclease. The VirD2 protein remains covalently attached to the 5′ terminus. The T-strand is coated by the single-strand binding protein VirE and the resulting complex is transferred to the plant cell. Although the mechanism by which the T-DNA complex is transported from the bacterium into the plant cell is not well understood, it is thought that the T-DNA complex leaves the Agrobacterium cell through a transmembrane structure consisting of proteins encoded by the virB operon. For extensive reviews on Agrobacterium tumefaciens transformation see Hooykaas and Schilperoort (Plant Molecular Biology 19 (1992) 15-38) and Hooykaas and Beijersbergen (Annu. Rev. Phytopathol. 32 (1994) 157-179). The ability of Agrobacterium tumefaciens to transfer its T-DNA into the plant cell, where it is stably integrated into the nuclear genome, has lead to a widespread use of this organism for gene transfer into plants and plant cells. In order to allow the regeneration of plants after Agrobacterium tumefaciens transformation the onc genes in the T-region have been deleted, which resulted in a disarmed or non-oncogenic T-DNA. Two types of vector systems have been developed for plant transformation. First a binary system, in which new genes are cloned in between the T-DNA borders of a plasmid containing an artificial T-DNA. This plasmid is subsequently introduced into an Agrobacterium strain harbouring a Ti plasmid with an intact vir region but lacking the T region (Hoekema et al.; Nature 303 (1983) 179-180 and Bevan; Nucl. Acids Res. 12 (1984) 8711-8721). Secondly a co-integrate system, in which new genes are introduced via homologous recombination into an artificial T-DNA already present on a Ti plasmid with an intact vir region (Zambryski et al.; EMBO-J. 2 (1983) 2143-2150).

A wide variety of plant species have been transformed using such systems. This includes many agriculturally important dicotyledonous species such as potato, tomato, soybean, sunflower, sugarbeet and cotton (for a review see Gasser and Fraley; Science 244, (1989) 1293-1299). Although Agrobacterium transformation of monocotyledonous plants seemed to be impossible for a long time, nowadays several species such as maize (Ishida et al.; Nature-Biotechnology 14 (1996) 745-750) and rice (Aldemita and Hodges; Planta 199 (1996) 612-617) have been transformed using Agrobacterium.

Another Agrobacterium species, Agrobacterium rhizogenes, possesses a similar natural gene transfer system, which is also contemplated by the present invention.

Transformation of micro-organisms using Agrobacterium

In addition to the many publications on transformation of plants using Agrobacterium tumefaciens, recently the results of some investigations on the use of Agrobacterium tumefaciens for transforming micro-organisms were published. Beijersbergen et al. (Science 256 (1992) 1324-1327) demonstrated that the virulence system of A. tumefaciens can mediate conjugative transfer between agrobacteria, which only relates to transformation of different strains of the same species. Bundock et al. (EMBO-J. 14 (1995) 3206-3214) reported on successful transformation of yeast by this soil bacterium. This result was subsequently confirmed by Piers et al. (Proc. Natl. Acad. Sci. USA, 93 (1996) 1613-1618). Both groups used DNA sequences from S. cerevisiae such as the yeast 2 mu. origin (Bundock et al.; EMBO-J. 14 (1995) 3206-3214) or yeast telomeric sequences and the ARS1 origin of replication (Piers et al.; Proc. Natl. Acad. Sci. USA, 93 (1996) 1613-1618) in order to stabilize the T-DNA in yeast. Risseeuw et al. (Mol. Cell. Biol. 16 (1996) 5924-5932) and Bundock & Hooykaas (Proc. Natl. Acad. Sci. USA, 93 (1996) 15272-15275) reported results on the mechanism of T-DNA integration in S. cerevisiae.

Plant biologists have modified the Ti plasmid to remove tumor-causing and superfluous genes but keep the genes necessary for T-DNA transfer and integration into nuclear DNA (Beijersbergen, A. et al., 1992, Science 256:1324-1327). In addition, binary vectors have been developed whereby the T-DNA region is harbored in trans from the rest of the Ti plasmid (Bevan, M. 1984, Nucleic Acids Res, 12:8711-8721). The binary vectors are smaller, can replicate in Escherichia coli, have selectable markers for growth in E. coli or plants, and provide cloning sites for addition of foreign DNA within the T-DNA. These binary vectors have been put to great use as insertional mutagens in plants and have been shown, with modification, to transfer T-DNA into S. cerevisiae yeast (Bundock, P. et al. 1995, EMBO J. 14:3206-3214), filamentous fungi (de Groot, M. J. et al., 1998. Nat. Biotechnol. 16:839-842). Changes necessary for use in fungi include addition of fungal selectable markers to the T-DNA and induction of the A. tumefaciens vir genes by special culture conditions; however, other modification would be known to those of skill in the art.

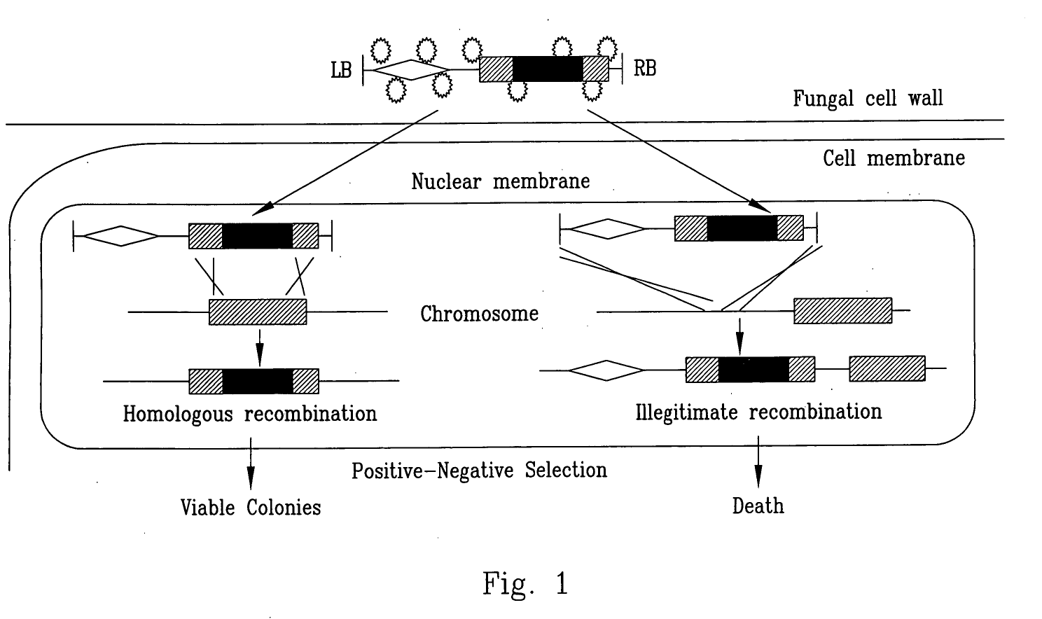

By way of overview and with reference to FIG. 1 which is a schematic diagram of ATMT-PNS, Agrobacterium tumefaciens, carrying a binary vector that contains a mutant allele (disrupted by a positive selection marker), and HSVtk on the T-DNA, are co-incubated with fungal cells in the presence of acetosyringone (AS), a chemical inducer of virulence genes of Agrobacterium tumefaciens. During co-cultivation, DNA situated between the left border (LB) and right border (RB) of the T-DNA is transported into fungal nuclei. Homologous recombination between the native gene and the mutant allele on the T-DNA leads to the loss of HSVtk. If the T-DNA integrates into a random location in the fungal genome via illegitimate recombination, both hph and HSVtk will be expressed. Gene KO mutants can be selected by subjecting transformants to both the positive and negative selection agents. A selection marker or marker generally encodes a polypeptide, which allows for maintenance of the plasmid in a population of cells. Some selection markers can also be used negatively in which loss of the marker confers viability to the host cells under certain growth conditions. Typical proteins include those that confer resistance to antibiotics or other toxins or allow growth in the presence of specific nutrients.

Markers for selection in fungi are well known to those of skill in the art and include those involved in growth on specific sugar, nucleoside, and amino acid substrates, such as trp, ura, leu, ade and his genes, which provide for maintenance of the plasmid in, for example, transformed yeast host cells lacking the corresponding functional genes on the host chromosome. Markers for selection in bacterial cells such as E. coli include those conferring resistance to antibiotics such as ampicillin, chloramphenicol, kanamycin, and the like. Positive markers contemplated by the present invention that are functional in fungal cells, particularly filamentous fungi include hygromycin B phosphotransferase (hph) gene, neomycin phosphotransferase (npt) gene, mutated beta-tublin (ben) gene, Bar, Ble, Sat-1, and cbx.

Generally, negative selection markers may code for enzymes, which convert nucleotide analogs to products which are lethal upon incorporation into DNA. More particularly, thymidine kinase (TK) is a versatile selection marker because cells can be selected for either loss or acquisition of this gene under different growth conditions. TK selection has proven useful for generation of cellular and viral gene knockouts. The presence of the thymidine kinase gene may be detected by the use of nucleoside analogs such as acyclovir, gancyclovir, or 5-fluoro-2′-deoxyuridine (F2dU) which will induce cytotoxic effects on cells that contain a functional thymidine kinase gene. The absence of sensitivity to these nucleoside analogs indicates the absence of the thymidine kinase gene.

This invention also contemplates use of screenable or scorable markers, which is a visual means for selecting transformants. Examples of scorable markers would include but are not limited to the coding sequence for green fluorescent protein (GFP) and the coding sequence for luciferase (LUX).

The present invention relates to a method of identifying and selecting transformants, termed ATMT-PNS, which is based on Agrobacterium-mediated transformation (ATMT) and a subsequent positive-negative selection scheme (PNS) to identify desired mutants. Employing two plant pathogenic fungi, Magnaporthe grisea and Fusarium oxysporum, this method proves potentially to be an efficient functional genomic tool for evaluating fungi. In its broadest sense the invention is characterized in that a cell is transformed with Agrobacterium comprising a vector containing a targeting construct wherein said construct comprises a first polynucleotide sequence encoding a negative selection marker linked to a fragment of DNA flanked by DNA sequences homologous to a polynucleotide to be targeted, wherein said DNA fragment is disrupted by a positive selection marker, under suitable conditions whereby recombination occurs wherein transformants resulting from a knockout lack a negative selection marker and ectopic, heterologous, or illegitimate transformants will express both a negative and a positive marker; and knockout mutants are selected by subjecting transformants to a positive and a negative selection agent.

Genetic transformation then occurs by simply incubating Agrobacterium with the host cell. During co-cultivation, DNA situated between the left border (LB) and right border (RB) of the T-DNA is transported into the host's nuclei. Homologous recombination between the native gene and the mutant allele on the T-DNA leads to the loss of the negative selection marker. If the T-DNA integrates into a random location in the fungal genome via illegitimate recombination, both the positive and negative selection marker is expressed. Knockout (KO) mutants can be selected by subjecting transformants to both a positive and a negative selection agent. Optionally, direct selection of putative knockout mutants may be performed by regenerating transformants in the presence of both a negative and positive selection agent.

The host organism can then be grown, and successfully transformed host cells can be selected using a subsequent positive-negative selection scheme as exemplified herein. A number of phylogenetically diverse fungi and fungus-like organisms may be used with minimal modifications, as host cells. Preferably, the fungi Magnaporthe grisea, Aspergillus fumigatus, Botrytis cineria, and Fusarium oxysporum are employed by the methods of the invention. More preferably, the fungi employed are Magnaporthe grisea and Fusarium oxysporum. Truncations of the negative selection are parameters that may be optimized to achieve desired marker selection or inhibition as is known to those of skill in the art and taught herein.

The following is a non-limiting general overview of molecular biology techniques that may be used in the invention.

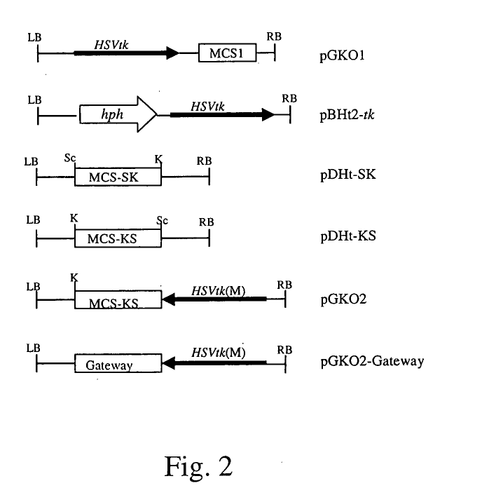

Schematics of the binary vectors of the invention are depicted in FIG. 2.

Targeting Constructs

The nucleic acid or targeting constructs of the present invention may be produced using standard methods known in the art. (See, e.g., Sambrook, et al., 1989, Molecular Cloning: A Laboratory Manual, Second Edition, Cold Spring Harbor Laboratory Press, Cold Spring Harbor, N.Y.; E. N. Glover (eds.), 1985, DNA Cloning: A Practical Approach, Volumes I and II; M. J. Gait (ed.), 1984, Oligonucleotide Synthesis; B. D. Hames & S. J. Higgins (eds.), 1985, Nucleic Acid Hybridization; B. D. Hames & S. J. Higgins (eds.), 1984, Transcription and Translation; B. Perbal, 1984, A Practical Guide To Molecular Cloning; F. M. Ausubel et al., 1994, Current Protocols in Molecular Biology, John Wiley & Sons, Inc.).

The targeting construct of the invention typically comprises a first polynucleotide sequence that is heterologous to the targeted sequence of interest, wherein the first polynucleotide sequence encodes a selectable marker which confers resistance to a drug or agent. The first polynucleotide sequence is linked to a fragment of DNA flanked by DNA sequences homologous to the gene to be targeted, wherein the DNA fragment is disrupted by a positive selection marker, which confers resistance to an antibiotic. The negative selection marker may be operatively linked to a promoter. It will be understood by one of skill in the art that virtually any promoter capable of driving this gene is suitable for the present invention. Many such promoters are available through sources such as GenBank. In a preferred embodiment the promoter is, but not limited to, the Cochliobolus heterostrophus glyceraldehyde-3-phosphate dehydrogenase (ChGPD) gene promoter. Synthetic promoters that regulate gene expression may also be used.

Selection Markers

The identification of the targeting event can be facilitated by the use of one or more selectable markers. A variety of selectable markers can be incorporated into the constructs disclosed herein. For example, a selectable marker which confers a selectable phenotype such as drug resistance, nutritional auxotrophy, resistance to a cytotoxic agent or expression of a surface protein, can be used. Selectable markers can be divided into two categories: positive selectable and negative selectable. In positive selection, cells expressing the positive selectable marker are capable of surviving treatment with a selective agent (such as hph and npt). In negative selection, cells expressing the negative selectable marker are destroyed in the presence of the selective agent. Positive selectable markers for use in a filamentous fungal host cell include, but are not limited to, hygromycin B phosphotransferase (hph) gene, neomycin phosphotransferase (npt) gene, mutated beta-tublin (ben) gene, Bar, Ble, Sat-1, and cbx, as well as equivalents thereof. Genes essential for the synthesis of an essential nutrient (such as amino acid arginine and nucleoside pyrimidine) may also be used as positive selection markers. To use such markers, the fungal strain to be transformed should have a mutation in these genes.

Additional candidate markers contemplated are gfp and luciferase (visual selection markers), URA3, a gene encoding orotidine-5-phosphate decarboxylase, and the Herpes Simplex Virus thymidine kinase (HSVtk) gene (conditional negative selection markers), and bacterial endotoxin genes (negative selection markers). It is to be understood that a selection marker may also be native to the host cell.

Targeted Sequences

According to the present invention, homologous recombination allows the preparation of constructs to target essentially any segment of the fungi, fungus-like organism, or other eukaryotic genome. The constructs of the present invention use a portion of the locus to be targeted. This approach is exemplified below with reference to particular polynucleotide sequence and particular fungal strains, however, the methods of the present invention are readily adaptable to other polynucleotide sequence and other species of fungi and other eukaryotic genomes.

The targeted sequence may be essential for cell growth under any conditions. Examples of specific phenotypes that may be screened for in the present method include but are not limited to, metabolic capacity (e.g., carbon source requirement, amino acid requirement, nitrogen source requirement, and nucleoside requirement); resistance to inorganic chemicals (e.g., acid, arsenate, azide, heavy metals, and peroxide); resistance to organic and biological chemicals (e.g., herbicides, fungicides, bactericidal agents, bacteriostatic agents, antibiotics, acridine, actinomycin, amino purine, amino phenylalanine, colicin, ethanol, fluoroacetate, mitomycin C, and nalidixic acid); resistance to biological agents (e.g., phages); resistance to physical extremes (e.g., temperature, pH, osmotolerance and radiation); enzymatic function (e.g., assays for protease, phosphatase, coagulase, urease, catalase, etc.); fatty acid composition; degradation; and hydrolysis. The phenotypes amenable to detection by the present invention are numerous and are contemplated by this invention.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usage and conditions.

Standard recombinant DNA and molecular cloning techniques used in the Examples are well known in the art and are described in Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Association and Wiley-Intersciences (1987).

Experimental protocol

Strains, media, and ATMT-PNS.

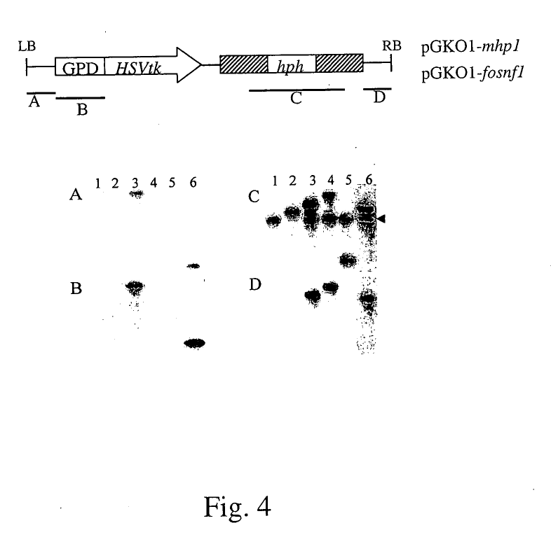

Agrobacterium tumefaciens strains AGL1 and EHA105 (Klee, 2000) were used to transform M. grisea strains KJ201 (Park et al., 2000), and 4091-5-8 (Valent et al., 1986), and F. oxysporum O-685 (Mullins et al., 2001). The following fungal and oomycete strains tested for sensitivity to F2dU were from the Inventors laboratory (Verticillium dahliae and Crinipellis perniciosa) or colleagues including David Geiser (Aspergillus oryzae, A. fumigatus, A. nidulans), Hye-Ji Kim (Thielaviopsis sp.), Wakar Uddin (Rhizoctonia solani), and Gary Moorman (Botrytis cineria, Pythium aphanidernatum, P. ultimum, P. irregulare, Phytophthora cactorum, and Phytophthora cinnamomi). Nucleoside analogs (Sigma), hygromycin B (Calbiochem), and geneticin (Sigma) were dissolved in water and filter-sterilized to prepare stock solutions and stored at −20° C. except hygromycin B (4° C.). For testing sensitivity to nucleoside analogs, M. grisea was grown on complete medium (Valent et al., 1986). Potato dextrose agar (Difco) was used for testing other fungi and oomycetes. ATMT was performed as previously described (Mullins et al., 2001). For measuring the efficiency of gene KO and the frequency of FP, transformants resistant to hygromycin B (250 μg/ml for M. grisea and 50 μg/ml for F. oxysporum) were transferred to a medium amended with 5 μM F2dU. Direct selection of putative gene KO mutants was carried out by regenerating transformants in the presence of both F2dU (5 μM or 50 μM) and hygromycin B. Different amounts of geneticin (800 μg/ml for M. grisea and 50 μg/ml for F. oxysporum) were utilized to determine the stability of neo. For determining the presence of HSVtk and gene KO in transformants, fungal genomic DNA was analyzed by PCR and/or Southern hybridization, using the probes shown in FIG. 4.

### Example 1

### Vector Construction

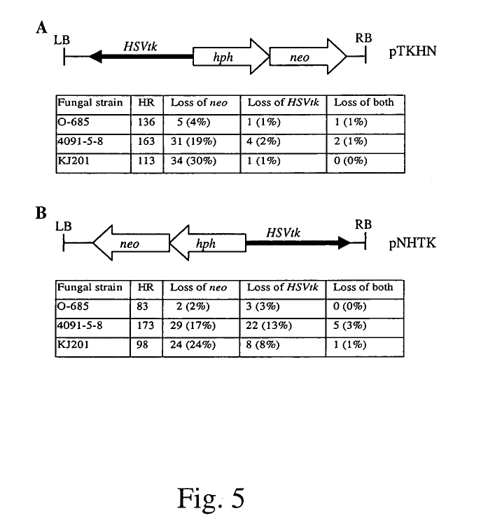

Schematic diagrams of the T-DNA of the binary vectors constructed in this study are shown in FIGS. 2 and 5. The ChGPD-HSVtk construct (1.8 kb EcoRI-HindIII fragment) in pGEM-3Zf (Promega) consists of three modules: the ChGPD promoter (0.5 Skb EcoRI-BamHI fragment), the open reading frame (ORF) of HSVtk (1.1 kb BamHI-SalI fragment), and the N. crassa β-tubulin gene terminator (0.2 kb SphI-HindIII fragment). Individual modules were constructed by PCR using a pair of primers containing appropriate restriction sites. All the modules were sequenced to verify their sequence.

Plasmid pBHt2-tk was constructed by cloning the 1.8 kb EcoRI-HindIII fragment carrying ChGPD-HSVtk between EcoRI and HindIII sites of pBHt2 (Mullins et al., 2001). To construct pGKO1, the 1.8kb EcoRI-HindIII fragment was made blunt by treating it with Klenow fragment in the presence of dNTPs, and cloned between the blunted XhoI and BstXI sites of pCAMBIA1300 (www.cambia.org.au). To produce pGKO1-fosnf1, a 1 kb fragment corresponding to FoSNF1 was amplified from F. oxysporum O-685 by PCR using the following primers: 5′-AGCACTAGTAATCTACCCGAGGCCAGTC-3′ (SEQ ID NO:1) and 5′-AGGCAATTGGGCGATTTTGACGTTGAGA-3′ (SEQ ID NO:2) (the underlined sequences correspond to SpeI and MfeI sites, respectively). After cloning the amplified fragment into pGEM-T Easy (Promega), a 56 bp HindIII-HindIII fragment of the amplified FoSNF1 was replaced with the 1.4 kb HpaI fragment of pBC1004 carrying hph, a gene encoding hygromycin B phosphotransferase (Carroll et al., 1994). The resulting mutant allele was digested with SpeI and MfeI and cloned between the EcoRI and XbaI sites of pGKO1 to produce pGKO1-fosnf1 (FIG. 4).

For vector pGKO1-mhp1, a 1.5 kb fragment containing MHP1 was amplified from M. grisea 70-15 by PCR using the following primers: 5′-ACGGAATTCTCGACATGGACCGTCTTG-3′ (SEQ ID NO:3) and 5′-AGCTCTAGAGTACCAAGCCGCACCACT-3′ (SEQ ID NO:4) (the underlined sequences correspond to EcoRI and XbaI sites, respectively). The hph gene was inserted into the blunted BglII site located in the middle of the amplified MHP1 locus to generate a mutant allele. The resulting mutant allele was digested with EcoRI and XbaI and cloned between the EcoRI and XbaI sites of pGKO1 to produce pGKO1-mhp1 (FIG. 4).

A 300 bp PvuII-PvuII fragment of pDHt (Mullins et al., 2001) containing MCS was replaced with a 0.8 kb HpaI-StuI fragment isolated from pGreenII0000 (Klee, 2000) to generate two binary vectors pDHt-KS and pDHt-SK (identical except the orientation of their MCS).

For constructing pNHTK and pTKHN, three selectable markers, neo (1.2 kb BamHI[-SalI fragment), hph (1.4 kb SalI-EcoRI fragment), and ChGPD-HSVtk (1.8 kb EcoRI-HindIII fragment), were initially cloned between BamHI and HindIII sites of pBluescript SK (Stratagene) in the order of SpeI-BamHI-neo-hph-ChGPD-HSVtk-HindIII, resulting in pSK1697. The 4.4 kb SpeI-HindIII fragment of pSK1697 was cloned between the Spel and HindIII sites of pDHt-SK and pDHt-KS to generate pNHTK and pTKHN, respectively (FIG. 5).

Selected restriction sites on the ChGPD-HSVtk construct in pGEM-3Zf were mutagenized using QuikChange Multi Site-Directed Mutagenesis kit (Stratagene) according to the manufacturer's instruction. The mutagenized ChGPD-HSVtk construct (as a blunted EcoRI-HindIII fragment) was cloned into a blunted SacI site of pDHt-KS, resulting in pGKO2. To allow for cloning of mutant allele into pGKO2 without relying on available restriction sites, we constructed pGKO2-Gateway as follows: the ccdB (control of cell death B) and chloramphenicol resistance genes flanked by the kattP sites in pDONR201 (Invitrogen) was amplified by PCR using the following primers: 5′-TCGCTCTAGAAATAATGATTTTATTTGAC-3′ (SEQ ID NO:5) and 5′-TCGCAAGCTTGCTGGATGGCAAATAATGAT-3′ (SEQ ID NO:6) (the underlined sequences correspond to XbaI and HindIII sites, respectively). The resulting product (2.3 kb) was first cloned in pGEM-T Easy for sequence verification and was subsequently cloned between the XbaI and HindIII sites of pGKO2.

### Example 2

### Herpes Simplex Virus Thymidine Kinase (HSVtk) Functions As A Negative Selection Marker In Diverse Fungi

A negative selection marker (a gene conferring lethality or easily discernable phenotype when expressed in transformants) flanking a mutant allele (generated by an insertion of a positive selection maker, such as the hygromycin B resistance gene) should allow quick identification of a target mutant without having to screen a large number of transformants by Southern or PCR (FIG. 1). Ectopic transformants will express both the negative and positive selection marker genes; while transformants resulted from gene KO should lack the negative selection marker.

Two genes were tested, one (Dtx-A) encoding diphtheria toxin subunit A, and the other (HSVtk) encoding a viral thymidine kinase, as potential negative selection markers for fungi. Although Dtx-A has been successfully utilized as a negative selection marker in plants (Czako and An, 1991; Terada et al., 2002), Dtx-A, expressed from two different fungal promoters, did not appear to be toxic to M. grisea and F. oxysporum (data not shown). The HSVtk gene product converts nucleoside analogs, such as Ganciclovir and 5-fluoro-2′-deoxyuridine (F2dU), to toxic compounds and has been shown to function as a conditional negative selection marker in diverse organisms (Capecchi, 1989; Sachs et al., 1997; Chen et al., 2002; Duraisingh et al., 2002).

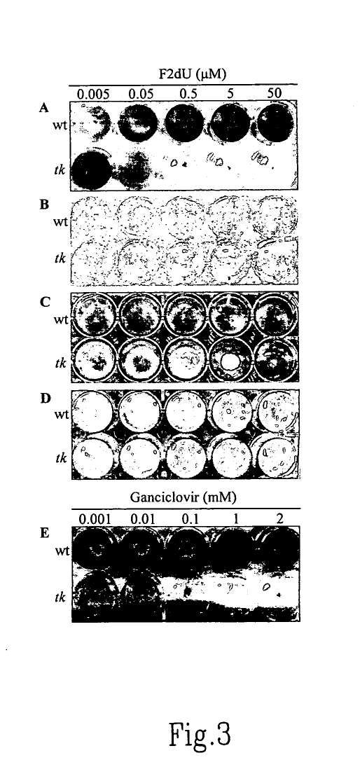

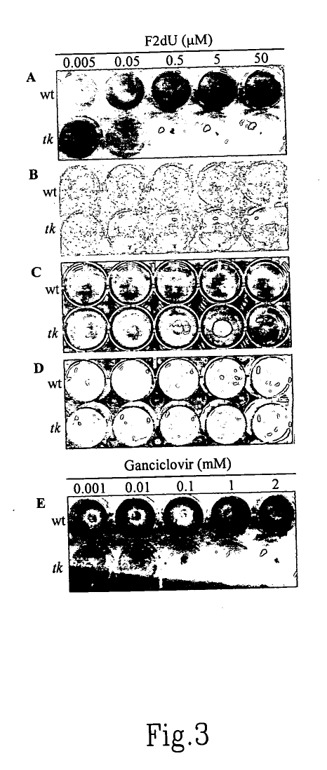

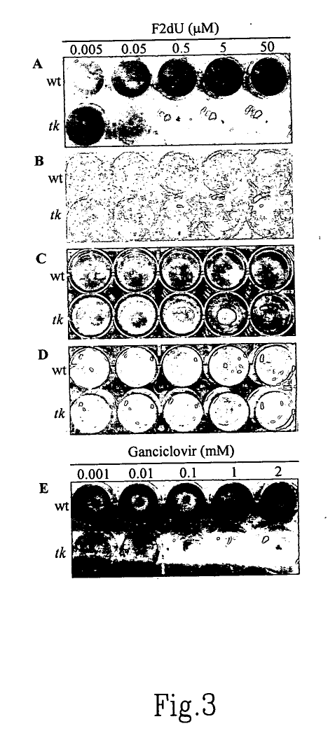

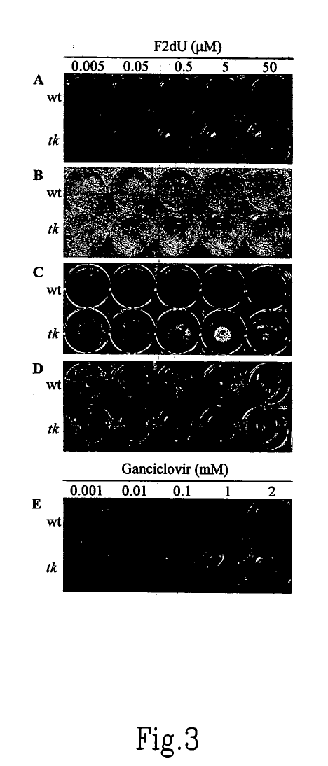

Transformants of Aspergillus fumigatus, Botrytis cineria, M. grisea and F. oxysporum generated using pBHt2-tk (FIG. 2), a binary vector carrying the HSVtk gene under the control of the Cochliobolus heterostrophus glyceraldehyde-3-phosphate dehydrogenase (ChGPD) gene promoter and the Neurospora crassa β-tubulin gene terminator on the T-DNA, exhibited sensitivity to Ganciclovir (with the exception of B. cineria) and F2dU but not to 5-fluoro-5′-deoxyuridine. Sensitivity to F2dU was much greater than that to Ganciclovir (FIG. 3 & not shown). For instance, the effective concentration of F2dU for completely blocking the growth of M. grisea was approximately 0.5 μM, while 1 mM Ganciclovir was needed to achieve the same degree of growth inhibition. Ganciclovir failed to inhibit the growth of B. cineria transformants even at 2 mM, while 5 nM of F2dU was sufficient to inhibit their growth (FIG. 3D). Transformants of A. fumigatus were much less sensitive to F2dU than were B. cineria, M. grisea and F. oxysporum transformants, requiring 50 μM F2dU for significant growth inhibition. In contrast, wild-type strains of these and other fungal and oomycete species, including ascomycetes (Aspergillus oryzae, A. nidulans, Thielaviopsis spp., and Verticillium dahliae), basidiomycetes (Rhizoctonia solani and Crinipellis perniciosa), and oomycetes (Pythium aphanidernatum, P. ultimum, P. irregulare, Phytophthora cactorum, and P. cinnamomi), did not exhibit sensitivity to F2dU or Ganciclovir at the concentrations that completely blocked the growth of HSVtk transformants (FIG. 3 & data not shown), suggesting the broad applicability of HSVtk as a negative selection marker.

### Example 3

### Mutagenesis of F. oxysporum and M. grisea genes via ATMT-PNS.

Two genes were utilized, F. oxysporum FoSNF1 (Ospina-Giraldo et al., 2003) and M. grisea MHP1 (a hydrophobin gene, unpublished result), to evaluate factors affecting the efficiency of gene knock-out (KO) via ATMT-PNS. To determine if bacterial strain-specific differences affected the efficiency of gene KO, we introduced gene disruption vectors pGKOl -fosnf1 and pGKO1-mhpl (FIG. 4) into two different A. tumefaciens strains, AGL1 and EHA105 (Klee, 2000). Two strains of M. grisea, KJ201 (Park et al., 2000) and 4091-5-8 (Valent et al., 1986), were also employed to evaluate fungal strain-specific differences. Hygromycin B-resistant transformants from two or more independent transformation experiments (multiple plates in each experiment) were pooled and analyzed for their sensitivity to F2dU and the presence of target mutation (Table 1).

TABLE 1
Analysis of transformants generated with pGKO1-fosnf1 and pGKO1-mhp1.

| Clones | Fungal | A. tumefaciens | | | | False positive |
|---|---|---|---|---|---|---|
| used | strain¹ | strain | HR² | FR³ | Gene KO⁴ | (FP)⁵ |
| pGKO1- | O-685 | AGL1 | 51 | 11( 22%) | 10 (20%) | 1 (9%) |
| fosnf1 | | EHA105 | 34 | 6 (18%) | 3 (9%) | 3 (50%) |
| pGKO1- | KJ201 | AGL1 | 70 | 26 (37%) | 18 (26%) | 8 (31%) |
| mhp1 | | EHA105 | 49 | 33 (67%) | 25 (51%) | 8 (24%) |
| | 4091-5-8 | AGL1 | 31 | 10 (32%) | 2 (6%) | 8 (80%) |
| | | EHA105 | 40 | 8 (20%) | 0 (0%) | 8 (100%) |

Fusarium oxysporum strain O-685 was transformed using pGKO1-fosnf1, and M. grisea strains KJ201 and 4091-5-8 were transformed using pGK01-mhp1.
²Total number of hygromycin B-resistant transformants isolated from two to four independent transformations (two plates for each transformation).
³The number and percentage of HR insensitive to 5 μM F2dU.
⁴The number and percentage of gene KO mutants among HRs.
⁵The number and percentage of FPs among FRs.

With F. oxysporum, AGL1 yielded a higher gene KO frequency than did EHA105 (20% vs. 9%). With M. grisea, AGL1 was better than EHA105 in generating gene KOs in 4091-5-8 (6% vs 0%), but produced fewer KOs in KJ201 than did EHA105 (26% vs 51%). With both AGLI and EHA105, the frequencies of gene KO in KJ201 was significantly higher than that in 4091-5-8. The MHPI allele used for mutagenesis was originally isolated from strain 70-15. Its sequence is identical to that from KJ201, but contains a number of polymorphic sites (31 out of 1540 bp) compared to that of 4091-5-8 (data not shown), suggesting that these polymorphisms might have led to the reduced gene KO frequency in 4091-5-8. Of course, it also is possible that 4091-5-8 has a less efficient homologous recombination machinery than KJ201.

In both species, certain fractions of F2dU-resistant transformants turned out to be false positive (FP; resistant to both hygromycin B and F2dU but lacking the target mutation). The frequency of FPs ranged from 9-50% in F. oxysporum to 24-100% in M. grisea (Table 1). In M. grisea, KJ201 yielded lower frequencies of FPs than did 4091-5-8. To determine whether FPs were caused by the truncation of HSVtk, we analyzed, via PCR and Southern hybridization, 28 FPs, three from F. oxysporum and 25 from M. grisea (FIG. 4 for examples). All FPs from F. oxysporum and KJ201 (16 in total), and 9 of 12 FPs from 4091-5-8 exhibited T-DNA truncation extending into the HSVtk ORF, but their RB region appeared intact (FIG. 4). One 4091-5-8 FP had extensive truncations at both the LB and RB. The remaining two FPs from 4091-5-8, however, had intact LB and ChGPD-HSVtk but were insensitive even to 50 μM F2dU (data not shown), suggesting that the expression of HSVtk was suppressed due to the chromosomal context of inserted T-DNA.

In addition to using the two-step selection described above, putative gene KO mutants were also directly selected by regenerating transformants from 0-685, KJ201, and 4091-5-8 in the presence of both hygromycin B and 5 μM F2dU. Unexpectedly, in all cases, the negative selection appeared leaky; a significant fraction of transformants (71%, 20%, and 82% in 0-685, KJ20, and 4091-5-8, respectively) exhibited sensitivity to F2dU when transferred to fresh media containing the same concentration of F2dU (data not shown). During the transformation procedure, following co-cultivation of fungal spores and A. tumefaciens cells on the membrane, a thick bacterial lawn is typically formed. When the membrane is transferred to the selective medium, which contains cefotaxime to kill the bacteria, in addition to hygromycin B and F2dU, A. tumefaciens cells begin to lyse. It was hypothesized that nucleosides released from the dead bacterial cells might have diluted the F2dU. If so, increasing the concentration of F2dU would reduce the leakiness of negative selection. However, even 50 μM F2dU appeared to only partially mitigated the leakiness; significant fractions of the O-685 and 4091-5-8 transformants were sensitive to 5 μM F2dU (Table 2). During this experiment, it was also noticed that in both fungi, the presence of 50 μM F2dU, but not 5 μM F2dU, consistently reduced (2 to 4 fold) the number of transformants relative to that generated in the presence of 0 or 5 μM F2dU, suggesting that too much F2dU might interfere with the regeneration of transformants.

TABLE 2
Leakiness of direct negative selection

| | Direct selection² | | F2dU-resistant |
|---|---|---|---|
| Fungal | F2dU | Number of | (FR) |
| strain¹ | (μM) | transformants | transformants³ |
| O-685 | 0 | 155 | 42 (27%) |
| | 5 | 128 | 38 (30%) |
| | 50 | 83 | 27 (33%) |
| 4091-5-8 | 0 | 138 | 78 (57%) |
| | 5 | 164 | 62 (38%) |
| | 50 | 41 | 31 (76%) |

¹O-685 and 4091-5-8 were transformed using pGKO1-fosnf1 and pGKO1-mhp1, respectively.
²Total number of transformants isolated from selection plates containing both hygromycin B and F2dU (0 μM, 5 μM or 50 μM). Seven plates were used for each treatment.
³The number and percentage of primary transformants resistant to 5 μM F2dU on a new plate containing 5 μM F2dU.

### Example 4

### Stability Of HSVtk Depends On Fungal Strains And The Location On The T-DNA

In pGKO1-fosnf1 and pGKO1-mhp1, HSVtk was located near the LB. To determine whether the RB side confers higher stability, we compared the stability of two markers, neo (a gene conferring resistance to geneticin) and HSVtk, at both the LB and RB sides. Following the isolation of hygromycin B-resistant transformants of F. oxysporum (O-685) and M. grisea (4091-5-8 and KJ201) using pNHTK and pTKHN (FIG. 5), we scored their resistance to F2dU and geneticin. Consistent with the data summarized in Table 1, at both locations, the stability of HSVtk and neo was significantly higher in F. oxysporum than in M. grisea (FIG. 5). While the stability of HSVtk was consistently higher at the RB than the LB in all the strains tested, there was no apparent difference for neo. In F. oxysporum O-685, the stability of neo was not significantly different from that of HSVtk, but in all both strains of M. grisea, neo was significantly more prone to inactivation than HSVtk at both locations.

### Example 5

### Construction Of New Vectors For ATMT-PNS

To facilitate gene KO, a number of new vectors were constructed (FIG. 2). The ChGPD-HSVtk construct on pGKO1 contains one or more of the following restriction sites: BamHI, EcoRV, PstI, Sad, Sal, and Smal. Most of these sites (except EcoRV) are also present in the MCS of pGKO1, thus significantly reducing the number of available sites for cloning mutant alleles for gene KO. These restriction sites were removed from the ChGPD-HSVtk construct via site-directed mutagenesis, and at the same time, codons were improved at the mutated sites based on the fungal codon usage (www.kazusa.orjp/codon). To further expand the number of available restriction sites for cloning in the previously developed pDHt vector (Mullins et al., 2001), the MCS in the vector was replaced with the one from pGreenII0000 (Hellens et al., 2000), resulting in pDHt-KS and pDHt-SK with 15 unique restriction sites. The mutated ChGPD-HSVtk construct was cloned at the SacI site in the MCS of pDHt-KS, generating pGKO2. To facilitate the disruption of a large number of genes, the GATEWAY™ system (Stratagene), designed to facilitate the movement of DNA fragments between vectors through the use of λ recombinase instead of restriction enzymes and ligase, was introduced into pGKO2, resulting in pGKO2-Gateway (FIG. 2).

Discussion

At present, >400 microbial genomes have been sequenced or sequencing projects are underway. Although just three fungal genomes have been published to date (Goffeau et al., 1996; Wood et al., 2002; Galagan et al., 2003), many more fungal genomes are currently being sequenced (http://wit.integratedgenomics.com/GOLD/). Considering that in many fungi, a major barrier in determining gene function via transformation-mediated gene KO has been the low efficiency of isolating mutants, development of techniques to circumvent this barrier is critical for effectively utilizing genome data to study fungal biology. ATMT exhibits several properties conducive to efficient gene manipulation in fungi, including high transformation efficiency, increased frequency of homologous recombination, and ability to transform spores and hyphae (Mullins and Kang, 2001). To further improve ATMT as a functional genomic tool for fungi, a negative selection scheme was incorporated that was originally developed to enhance gene KO efficiency in animal cells (Capecchi, 1989). This technique, termed ATMT-PNS, exhibits potential as an efficient, universal functional genomic tool for harnessing the growing body of fungal genome sequence data to study the molecular basis of fungal biology.

A strategy similar to ATMT-PNS was recently applied to enhance the efficiency of gene KO in Neurospora crassa (Pratt and Aramayo, 2002). While this technique allowed a significant enrichment of gene KO mutants, its utility was limited because the negative selection marker used, the mat α-1 gene, confers toxicity only to N. crassa. In contrast, HSVtk can function as a universal, conditional negative selection marker. Our survey strongly suggests the lack of an enzyme equivalent to HSVtk in most fungi and oomycete. Only a wild-type strain of B. cineria exhibited noticeable sensitivity to F2dU (FIG. 3). In addition to the four fungal species tested in our study (FIG. 3), transformants of N. crassa (Sachs et al., 1997; Pratt and Aramayo, 2002) and the human pathogenic basidiomycete Cryptococcus neoformans (Y. Chang and J. Kwon-Chung at NIH, personal communication) that express HSVtk also exhibited sensitivity to F2dU. Given that diverse fungi have now been successfully transformed via ATMT (de Groot et al., 1998; Gouka et al., 1999; Abuodeh et al., 2000; Chen et al., 2000; Covert et al., 2001; Malonek and Meinhardt, 2001; Zwiers and De Waard, 2001; Hanif et al., 2002; Sullivan et al., 2002; Campoy et al., 2003; Combier et al., 2003; Zhang et al., 2003), the binary vectors developed and disclosed herein can be utilized to disrupt genes in many fungi; the only modification that might be needed in certain fungi would be to replace the ChGPD promoter with an appropriate promoter for target fungi. Due to the modular structure of the negative selection marker, such a modification should be simple.

There are two problems that could potentially limit the efficiency of ATMT-PNS, one of which is the leakiness of the negative selection during the regeneration of transformants. Considering that even 50 μM F2dU failed to select against F2dU-sensitive transformants during regeneration, it seems unlikely that nucleosides released from dead A. tumefaciens cells are responsible for the leakiness. Alternatively, the expression of HSVtk driven by the ChGPD promoter might be suppressed during regeneration. If so, using a different fungal promoter might solve the problem. However, screening transformants for their sensitivity to F2dU after their regeneration is a solution to this problem. Another problem is the appearance of FP. Although even in the presence of FP, the negative selection facilitated the rapid identification of gene KO mutants in F. oxysporum and M. grisea by eliminating most ectopic transformants (Table 1), in fungi that exhibit both a high rate of T-DNA truncation/inactivation and a low gene KO frequency, the problem caused by FP can be compounded. To reduce the frequency of FP, new binary vectors, pGKO2 and pGKO2-Gateway (FIG. 2) were constructed. When HSVtk was located near the RB, the frequency of its loss (or inactivation) was significantly lower (ranging from <1% in F. oxysporum to 2% in M. grisea 4091-5-8) than that near the LB (ranging from 4% in F. oxysporum to 13% in M. grisea 4091-5-8), suggesting that gene KO via the use of pGKO2 or pGKO2-Gateway should significantly reduce the frequency of FP.

Considering that the gene KO efficiency and the frequency of FP potentially depended on A. tumefaciens strains and fungal species/strains (Table 1 and FIG. 5), for a new fungal species to be mutagenized via ATMT-PNS, evaluating different combinations of these factors prior to launching a large-scale gene KO experiment is recommended.

References

Abuodeh, R. O., Orbach, M. J., Mandel, M. A., Das, A., and Galgiani, J. N. (2000). Genetic transformation of Coccidioides immitis facilitated by Agrobacterium tumefaciens. J. Infect. Dis. 181, 2106-2110.
Askenazi, M., Driggers, E. M., Holtzman, D. A., Norman, T. C., Iverson, S., Zimmer, D. P., Boers, M.-E., Blomquist, P. R., Martinez, E. J., Monreal, A. W., Feibelman, T. P., Mayorga, M. E., Maxon, M. E., Sykes, K., Tobin, J. V., Cordero, E., Salama, S. R., Trueheart, J., Royer, J. C., and Madden, K. T. (2003). Integrating transcriptional and metabolite profiles to direct the engineering of lovastatin-producing fungal strains. Nat. Biotech. 21, 150-156.
Campoy, S., Perez, F., Martin, J. F., Gutierrez, S., and Liras, P. (2003). Stable transformations of the azaphilone pigment-producing Monascus purpureus obtained by protoplast transformation and Agrobacterium-mediated DNA transfer. Curr. Genet. 43, 447-452.
Capecchi, M. R. (1989). Altering the genome by homologous recombination. Science 244, 1288-1292.
Carroll, A. M., Sweigard, J. A., and Valent, B. (1994). Improved vectors for selecting resistance to hygromycin. Fungal Genet. Newsl. 41, 22.
Chen, S., Hong, Y., and Schartl, M. (2002). Development of a positive-negative selection procedure for gene trageting in fish cells. Aquaculture 214, 67-79.
Chen, X., Stone, M., Schlagnhaufer, C., and Romaine, C. P. (2000). A fruiting body tissue method for efficient Agrobacterium-mediated transformation of Agaricus bisporus. Appl. Environ. Microbiol. 66, 4510-4513.
Combier, J.-P., Melayah, D., Raffier, C., Gay, G., and Marmeisse, R. (2003). Agrobacetrium tumefaciens-mediated transformation as a tool for insertional mutagenesis in the symbiotic ectomycorrhizal fungus Hebeloma cylindrosporum. FEMS Microbiol. Lett. 220, 141-148.
Covert, S. F., Kapoor, P., Lee, M., Briley, A., and Nairn, C. J. (2001). Agrobacterium tumefaciens-mediated transformation of Fusarium circinatum. Mycol. Res. 105, 259-264.
Czako, M., and An, G. (1991). Expression of DNA coding for diphtheria toxin chain A is toxic to plant cells. Plant Physiol. 95, 687-692.
de Groot, M. J. A., Bundock, P., Hooykaas, P. J. J., and Beijersbergen, A. G. M. (1998). Agrobacterium tumefaciens-mediated transformation of filamentous fungi. Nature Biotech. 16, 839-842.
Demain, A. L. (2000). Microbial biotechnology. Trends Biotech. 18, 26-31. Duraisingh, M. T., Triglia, T., and Cowman, A. F. (2002). Negative selection of Plasmodium falciparum reveals targeted gene deletion by double crossover recombination. Int. J. Parasitol. 32, 81-89.
Galagan, J. E., Calvo, S. E., Borkovich, K. A., Selker, E. U., Read, N. D., Jaffe, D., FitzHugh, W., Ma, L.-J., Smimov, S., Purcell, S., Rehman, B., Elkins, T., Engels, R., Wang, S., Nielsen, C. B., Butler, J., Endrizzi, M., Qui, D., Ianakiev, P., Bell-Pedersen, D., and Nelson, M. A. (2003). The genome sequence of the filamentous fungus Neurospora crassa. Nature 422, 859-867.
Goffeau, A., Barrell, B. G., Bussey, H., Davis, R. W., Dujon, B., Feldman, H., Galibert, F., Hoheisel, J. D., Jacq, C., Johnston, M., and al., e. (1996). Life with 6000 genes. Science 274, 546-567.
Gouka, R. J., Gerk, C., Hooykaas, P. J. J., Bundock, P., Musters, W., Verrips, C. T., and de Groot, M. J. A. (1999). Transformation of Aspergillus awamori by Agrobacterium tumefaciens-mediated hohologous recombination. Nat. Biotechnol. 17, 598-601.
Hanif, M., Pardo, A. G., Gorfer, M., and Raudaskoski, M. (2002). T-DNA transfer and integration in the ectomyrrhizal fungus Suillus bovinus using hygromycin B as a selectable marker. Curr. Genet. 41, 183-188.
Hawksworth, D. L. (1991). The fungal dimension of biodiversity: magnitude, significance, and conservation. Mycol. Res. 95, 641-655.
Hellens, R. P., Edwards, E. A., Leyland, N. R., Bean, S., and Mullineaux, P. M. (2000). pgreen: a versatile and flexible binary vector for Agrobacterium-mediated plant transformation. Plant Mol. Biol. 42, 819-832.
Hudler, G. W. (1998). Magical mushrooms, mischievous molds. (Princeton: Princeton University Press).
Klee, H. (2000). A guide to Agrobacterium binary Ti vectors. Trends Plant Sci. 5, 446-451.
Malonek, S., and Meinhardt, F. (2001). Agrobacterium tumefaciens-mediated genetic transformation of the phytopathogenic ascomycete Calonectria morganii. Curr. Genet. 40, 152-155.
Mullins, E., Romaine, C. P., Chen, X., Geiser, D., Raina, R., and Kang, S. (2001). Agrobacterium tumefaciens-mediated transformation of Fusarium oxysporum: An efficient tool for insertional mutagenesis and gene transfer. Phytopathology 91, 173-180.
Mullins, E. D., and Kang, S. (2001). Transformation: A tool for studying fungal pathogens of plants. Cell. Mol. Life Sci. 58, 2043-2052.
Ospina-Giraldo, M., Mullins, E., and Kang, S. (2003). Loss of function of the Fusarium oxysporum SNF1 gene reduces virulence on cabbage and Arabidopsis. Curr. Genet. 44, 49-57.
Park, S. G., Kim, S. O., Koh, H. J., and Lee, Y. H. (2000). A blast lesion mimic mutant of rice. In Advances in Rice Blast, D. Tharreau, M. H. Lebrun, N. J. Talbot, and J. L. Notteghem, eds (Dordrecht: Kluwer Academic Press), pp. 79-85.
Pratt, R. J., and Aramayo, R. (2002). Improving the efficiency of gene replacements in Neurospora crassa: a first step towards a large-scale functional genomics project. Fungal Genet. Biol. 37, 56-71.
Sachs, M. S., Selker, E. U., Lin, B., Roberts, C. J., Luo, Z., Vaught-Alexander, D., and Margolin, B. S. (1997). Expression of herpes virus thymidine kinase in Neurospora crassa. Nucl. Acids Res. 12, 2389-2395.
Sullivan, T. D., Rooney, P. J., and Klein, B. S. (2002). Agrobacterium tumefaciens integrates transfer DNA into single chromosomal sites of dimorphic fungi and yields homokaryotic progeny from multinucleate yeast. Eukaryotic Cell 1, 895-905.
Terada, R., Urawa, H., Inagaki, Y., Tsugane, K., and lida, S. (2002). Efficient gene targeting by homologous recombination in rice. Nat. Biotech. 20, 1030-1034.
Valent, B., Crawford, M. S., Weaver, C. G., and Chumley, F. G. (1986). Genetic studies of fertility and pathogenicity in Magnaporthe grisea (Pyricularia oryzae). Iowa State J. Res. 60, 569-594.
Wood, V., Gwilliam, R., Rajandream, M.-A., Lyne, M., Stewart, A., Sgouros, J., Peat, N., Hayles, J., Baker, S., Basham, D., Bowman, S., Brooks, K., Brown, D., Brown, S., Chillingworth, T., Churcher, C., Collins, M., Connor, R., Connor, R., Cronin, A., Davis, P., and Feltwell, T. (2002). The genome sequence of Schizosaccharomyces pombe. Nature 415, 871-880.
Zhang, A., Lu, P., Dahl-Roshak, A. M., Paress, P. S., Kennedy, S., Tkacz, J. S., and An, Z. (2003). Efficient disruption of a polyketide synthase gene (pks1) required for melanine synthesis through Agrobacterium-mediated transformation of Glarea lozoyensis. Mol. Gen. Genomics 268, 645-655.
Zwiers, L.-H., and De Waard, M. A. (2001). Efficient Agrobacterium tumefaciens-mediated gene disruption in the phytopathogen Mycosphaerella graminicola. Curr. Genet. 39, 388-393.

1. A method of identifying and selecting transformants comprising;
transforming a host cell with Agrobacterium under suitable conditions whereby recombination occurs, the Agrobacterium comprising a vector containing a targeting construct wherein said construct comprises a first polynucleotide sequence encoding a negative selection marker linked to a fragment of DNA flanked by DNA sequences homologous to a polynucleotide to be targeted, wherein said DNA fragment is disrupted by a positive selection marker; and
selecting transformants by subjecting a transformed host cell to a positive and a negative selection agent.

2. The method of claim 1, wherein transformants resulting from a knockout lack a negative selection marker and ectopic, heterologous, or illegitimate transformants express both a negative and a positive selection marker.

3. The method of claim 1, wherein said cell is a fungal cell.

4. The method of claim 3, wherein said fungal cell comprises mycelial fragments, spores, and protoplasts.

5. The method of claim 1, wherein said negative selection marker confers susceptibility to an agent.

6. The method of claim 5, wherein said negative selection marker is operably linked to a promoter sequence.

7. The method of claim 5, wherein said negative selection marker is selected from the group consisting of a herpes simplex virus thymidine kinase (HSVtk), and a bacterial endotoxin gene.

8. The method of claim 7, wherein said negative selection marker is HSVtk.

9. The method of claim 1, wherein said positive selection marker confers resistance to an antibiotic.

10. The method of claim 9, wherein said positive selection marker is selected from the group consisting of hygromycin B phosphotransferase (hph) gene, neomycin phosphotransferase (npt) gene, mutated beta-tublin (ben) gene, Bar, Ble, Sat-1, and cbx.

11. The method of claim 10, wherein said positive selection marker is a hygromycin resistance gene (hph).

12. The method of claim 3, wherein said fungal cell is a fungal species selected from the group consisting of Aspergillus fumigatus, Botrytis cineria, Magnaporthe grisea and Fusarium oxysporum.

13. The method of claim 12, wherein said fungal cell is Magnaporthe grisea.

14. The method of claim 12, wherein said fungal cell is Fusarium oxysporum.

15. The method of claim 1, wherein said transformation is mediated by Agrobacterium tumefaciens.

16. A strain of fungal cells transformed by the method of claim 1.

17. A polynucleotide construct comprising a first polynucleotide sequence encoding a negative selection marker linked to a fragment of DNA flanked by DNA sequences homologous to a polynucleotide to be targeted, wherein said DNA fragment is disrupted by a positive selection marker.

18. A vector comprising the polynucleotide construct of claim 17.

19. The vector of claim 18 capable of transforming fungal cells in culture susceptible to infection by Agrobacterium tumefaciens.

20. An Agrobacterium tumefaciens cell comprising the vector of claim 18.

21. A method of identifying a gene knockout mutant comprising:
(a) providing a polynucleotide construct comprising a first polynucleotide sequence that encodes a negative selection marker linked to a fragment of DNA flanked by DNA sequences homologous to the polynucleotide to be targeted, wherein said DNA fragment is disrupted by a positive selection marker;
(b) introducing into Agrobacterium the construct provided in (a), thereby producing a resultant Agrobacterium cells containing a DNA fragment with a disrupted sequence;
(c) incubating Agrobacterium produced in (b) with fungal cells under conditions so that T-DNA containing said construct is integrated into a fungal cell genome, wherein transformants resulting from knockout lack a negative selection marker and ectopic, heterologous, or illegitimate transformants express both a negative and a positive selection marker; and
(d) selecting knockout mutants by subjecting transformed fungal cells to a positive and a negative selection agent.

22. The method of claim 21, wherein said DNA fragment is a gene of interest that is rendered nonfunctional by insertion of a selection marker, thereby generating a null mutation to assess a phenotypic affect of at least one mutant allele.

23. The method of claim 21, wherein said fungal cells comprise mycelial fragments, spores, and protoplasts.

24. The method of claim 21, wherein said negative selection marker is operably linked to a promoter sequence.

25. The method of claim 21, wherein said positive selection marker is selected from the group consisting of hygromycin B phosphotransferase (hph) gene, neomycin phosphotransferase (npt) gene, mutated beta-tublin (ben) gene, Bar, Ble, Sat-1, and cbx.

26. The method of claim 25, wherein said positive selection marker is a hygromycin resistance gene.

27. The method of claim 21, wherein said negative selection marker is selected from the group consisting of herpes simplex virus thymidine kinase (HSVtk), a bacterial endotoxin gene, and a diphtheria toxin A fragment.

28. The method of claim 27, wherein said negative selection marker is HSVtk.

29. The method of claim 21, wherein said negative selection agent is selected from the group consisting of ganciclovir, acyclovir, and 5-fluoro-2′-deoxyuridine (F2dU).

30. The method of claim 29, wherein said negative selection agent is 5-fluoro-2′-deoxyuridine (F2dU).

31. The method of claim 21, wherein said positive selection agent is selected from the group consisting of hygromycin B, geneticin or G-418, benomyl, basta, phleomycin, nourseothricin, and carboxin.

32. The method of claim 31, wherein said positive selection agent is hygromycin B.

33. The method of claim 21, wherein said fungal cells are fungal species selected from the group consisting of Aspergillus fumigatus, Botrytis cineria, Magnaporthe grisea and Fusarium oxysporum.

34. The method of claim 33, wherein said fungal cells are Magnaporthe grisea.

35. The method of claim 33, wherein said fungal cells are Fusarium oxysporum.

36. A strain of fungal cells transformed by the method of claim 21.

37. A method of transforming fungal cells to identify mutants comprising:
inserting a polynucleotide construct to be introduced into fungal cells into an Agrobacterium-based vector between T-DNA borders in that vector;
introducing said vector containing said DNA construct into Agrobacterium tumefaciens cells, wherein said cells contain a virulence region in its DNA;
inducing virulence genes to T-DNA containing said construct from said Agrobacterium tumefaciens and incubating said Agrobacterium tumefaciens with a fungal cells to be transformed; and
selecting transformed fungal cells from untransformed fungal cells by subjecting transformants to a positive and a negative selection agent.

38. The method of claim 37, wherein said fungal cells comprise mycelial fragments, spores, and protoplasts.

39. The method of claim 37, wherein said polynucleotide construct comprises a disruption cassette.

40. The method of claim 39, wherein said cassette comprises a DNA fragment having at least one mutant allele, wherein said mutant allele is generated by the insertion of a positive selection marker.

41. The method of claim 37, wherein said construct further comprises a negative selection marker that is operably linked to a promoter sequence.

42. The method of claim 40, wherein said positive selection marker is selected from the group consisting of hygromycin B phosphotransferase (hph) gene, neomycin phosphotransferase (npt) gene, mutated beta-tublin (ben) gene, Bar, Ble, Sat-1, and cbx.

43. The method of claim 42, wherein said positive selection marker is a hygromycin resistance gene.

44. The method of claim 37, wherein said negative selection marker is selected from the group consisting of herpes simplex virus thymidine kinase (HSVtk), a bacterial endotoxin gene, and a diphtheria toxin A fragment.

45. The method of claim 44, wherein said negative selection marker is HSVtk.

46. The method of claim 37, wherein said negative selection agent is selected from the group consisting of ganciclovir, acyclovir, and 5-fluoro-2′-deoxyuridine (F2dU).

47. The method of claim 46, wherein said negative selection agent is 5-fluoro-2′-deoxyuridine (F2dU).

48. The method of claim 32, wherein said positive selection agent is selected from the group consisting of hygromycin B, geneticin or G-418, benomyl, basta, phleomycin, nourseothricin, and carboxin.

49. The method of claim 48, wherein said positive selection agent is hygromycin B.

50. The method of claim 37, wherein said fungal cells are fungal species selected from the group consisting of Aspergillus fumigatus, Botrytis cineria, Magnaporthe grisea and Fusarium oxysporum.

51. The method of claim 50, wherein said fungal cells are Magnaporthe grisea.

52. The method of claim 50, wherein said fungal cells are Fusarium oxysporum.

53. A strain of fungal cells transformed by the method of claim 37.

54. A method of identifying and selecting transformants comprising:
transforming fungal cells with Agrobacterium tumefaciens under suitable conditions whereby recombination occurs, wherein transformants resulting from a gene knockout lack a negative selection marker and ectopic, heterologous, or illegitimate transformants will express a negative and a positive selection marker, said Agrobacterium tumefaciens comprising a gene disruption vector, said vector comprises a polynucleotide encoding a negative selection marker linked to a fragment of DNA flanked by DNA sequences homologous to the polynucleotide to be targeted, wherein said fragment contains at least one mutant allele, wherein said mutant allele is generated by the insertion of a positive selection marker;
regenerating transformants in the presence of both a positive and a negative selection agent; and
selecting putative knockout mutants.

55. The method of claim 54, wherein said fungal cells comprise mycelial fragments, spores, and protoplasts.

56. The method of claim 54, wherein said fungal cells are fungal species selected from the group consisting of Aspergillus fumigatus, Botrytis cineria, Magnaporthe grisea and Fusarium oxysporum.

57. The method of claim 56, wherein said fungal cells are Magnaporthe grisea.

58. The method of claim 56, wherein said fungal cells are Fusarium oxysporum.

59. A strain of fungal cells transformed by the method of claim 54.

60. A method of identifying and selecting transformants comprising: transforming fungal cells with Agrobacterium tumefaciens cells under suitable conditions whereby recombination occurs wherein transformants resulting from gene knockout lack a negative selection marker and ectopic, heterologous, or illegitimate transformants express both a negative and a positive marker, said Agrobacterium tumefaciens cells comprising a gene disruption vector, said vector comprising in an operable orientation a pgreen II cloning site, a polynucleotide sequence that encodes a negative selection marker, said sequence is linked to a fragment of DNA, wherein said DNA fragment is disrupted by a positive selection marker; and selecting gene knockout mutants by subjecting transformed fungal cells to a positive and a negative selection agent.

61. The method of claim 60, wherein said fungal cells are fungal species selected from the group consisting of Magnaporthe grisea and Fusarium oxysporum.

62. A targeted polynucleotide having undergone homologous recombination with the vector of claim 1 so as to incorporate said DNA fragment disrupted by a positive selectable marker into said targeted polynucleotide.

63. A polynucleotide construct in an operable orientation comprising a first polynucleotide sequence encoding a negative selection marker; a DNA fragment disrupted by a positive selection marker; and a pGreen II cloning site.

64. The polynucleotide construct of claim 17, wherein said first polynucleotide sequence a herpes simplex virus thymidine kinase (HSVtk) and said second polynucleotide sequence disrupted by an hygromycin resistance selection marker.

65. The polynucleotide construct of claim 17, wherein said second polynucleotide is homologous to a targeted polynucleotide sequence in a fungal host cell.
